(19) United States
(12) Patent Application Publication
(10) Pub. No.: US 2024/0175795 A1
(43) Pub. Date: May 30, 2024

(54) A FLOW CELL AND USE THEREOF

(71) Applicant: PLANKTON ANALYTICS LIMITED, Plymouth Devon (GB)

(72) Inventors: Philip Frederick Culverhouse, Plymouth Devon (GB); Julian Bernard Tilbury, Plymouth Devon (GB)

(73) Assignee: PLANKTON ANALYTICS LIMITED, Plymouth Devon (GB)

(21) Appl. No.: 18/552,529

(22) PCT Filed: Apr. 7, 2022
(86) PCT No.: PCT/GB2022/050868

§ 371 Date: Sep. 26, 2023

(30) Foreign Application Priority Data
Apr. 8, 2021 (GB) 2104989.5

(51) Int. Cl. G01N 15/0227; G01N 15/14; G01N 15/1433

(52) U.S. Cl. CPC G01N 15/0227; G01N 15/1433; G01N 2015/1479

(57) ABSTRACT

A flow cell for the analysis of objects in a liquid stream is provided, the flow cell comprising: a flow cell body having a chamber therein defined by an inner surface of the flow cell body, the flow cell body having an inlet end and an outlet end; the inlet end of the flow cell body being provided with a first fluid coupling member having a flow passage therethrough; the outlet end of the flow cell body being provided with a second fluid coupling member having a flow passage therethrough; the flow cell body comprising a first transparent portion, through which light may enter the chamber to illuminate objects within the chamber, and a second transparent portion through which objects within the chamber may be imaged; wherein the chamber comprises a first transition portion adjacent the inlet end of the flow cell body, the first transition portion comprising a smooth transition between the flow passage of the first fluid coupling member and the inner surface of the flow cell body defining the chamber; and wherein the chamber comprises a second transition portion adjacent the outlet end of the flow cell body, the second transition portion comprising a smooth transition between the flow passage of the second fluid coupling member and the inner surface of the flow cell body defining the chamber. An imaging apparatus comprising the flow cell is also provided. The flow cell and imaging apparatus find use in imaging such objects as plankton, detritus and bubbles in liquid streams.

The present invention relates to a flow cell for use in the analysis of samples of a liquid, in particular water samples. The present invention also relates to an analyser comprising the flow cell and aspects thereof.

The analysis of samples of a liquid, in particular water samples, is required for many reasons. One particular need for the analysis of water samples arises in the study and monitoring of plankton in marine and freshwater environments. An understanding of the diversity and distribution of plankton in the seas and oceans of the world is important for many reasons, including identifying the response of the ecosystem to climate change. Plankton are sensitive to changes in physical aspects of their environment, such as temperature of the water. As a result, marine plankton are sensitive to environmental changes of the world's oceans and, if analysed, can provide a means of determining climate change on a global scale. Further, plankton lie between algae and fish larvae in the marine food chain. As a result, an analysis of plankton is useful in the modelling of food-webs and monitoring fish stocks in the seas and oceans. Analysis of plankton is also useful in the detection of harmful algal blooms in coastal waters.

The need for a system for the automatic analysis of plankton in marine environments is discussed by Culverhouse P. F. et al., ‘Automatic Image Analysis of Plankton: Future Perspectives’, Marine Ecology Progress Series, Vol. 312, 2006, pages 297 to 309. In particular, it is suggested that the rapid, automatic identification and categorisation of species of plankton in an oceanographic region is an important step in meeting the demands for plankton analysis.

Systems for the analysis of plankton are known in the art. For example, an integrated plankton and imaging instrument is described by Culverhouse P. F. et al., ‘HAB Buoy: A New Instrument For In Situ Monitoring and Early Warning of HAB Events’, African Journal of Marine Science, 2006, 28(2), pages 245 to 250. The instrument combines a high speed camera for image acquisition with software for labelling specimens. A development of this instrument is described by Culverhouse P. F. et al., ‘An Instrument For Rapid Mesozooplankton Monitoring At Ocean Basin Scale’, Journal of Marine Biology and Aquaculture, 2015, 1(1), pages 1 to 11.

CN202421056U discloses a rapid plankton monitoring device, which comprises a detection cell, a detection passage, a filter, an objective and a charge coupled device.

JP2007309819A discloses an apparatus and a method for observing plankton. Some plankton taxa having a phototaxis are made to flow in a cell for observation. The cell has a retaining region in which test water is retained. Light is emitted into the cell, causing the plankton to migrate in the direction of the incident light.

An in-flow instrument for imaging and identifying meso-zooplankton from a ship's clean pumped sea water supply is described by Culverhouse P. F. et al., ‘Ocean-Scale Monitoring Of Mesozooplankton On Atlantic Meridonial Transect 21’, Journal of Marine Biology and Aquaculture, 2016, 2(1), pages 1 to 13. The instrument allows for the monitoring of plankton from a vessel while the vessel is underway.

More recently, CN107194403 discloses a plankton particle size spectrum detection system and a method thereof. The system comprises an image acquisition processing unit, a target detection unit, an edge information extraction unit, and a counting unit that are sequentially connected.

CN206990427U discloses a plankton polarization imager.

CN208255038U discloses an image device for the analysis of plankton. The device comprises a fluid module having a sample pump, a module for forming images and a control module.

Still more recently, CN209784139U discloses a scanning, focusing, plankton flow type counting system for use in the detection of plankton.

CN210269629U discloses a water surface plankton detection device. The detection device comprises a sampling system and a detection system.

CN210294046U discloses a mobile detector for the in-situ, rapid, quantitative detection of plankton.

In addition to the analysis of plankton in marine water, there are other aspects to the analysis of water samples. In particular, there is a need for the detection and analysis of bubbles in water, for example sea water. Small bubbles in water have the potential to supersaturate the water with gases. However, the analysis of small bubbles, for example having a diameter of from 100 μm to 3 mm, is very challenging.

A number of systems and methods for detecting the presence of bubbles in a liquid stream are known in the art. An optical bubble detection system is described and shown in U.S. Pat. No. 6,531,708. The system comprises a flow cell and an optical sensor. Light refraction is used to detect the presence of a bubble in the flow cell.

WO 2007/022052 discloses a method for detecting the presence of bubbles in a flow meter.

US 2009/293588 discloses an air bubble detector for use in the medical field, in particular in the delivery of liquid to patient through tubing.

While the prior art systems and methods described above may provide for the detection of a bubble in a liquid stream, they do not provide for the analysis of the bubbles.

US 2021/0010919 discloses an optical flow measuring cell and a measuring device comprising the cell. The cell is for investigating a fluid flow.

WO 2019/202132 discloses an apparatus and method for fluid analysis. The apparatus and method find use in analysing particles and contaminants present in a fluid, for example in a lubricating oil.

A concentration meter for use in measuring paper pulp consistency is disclosed in WO 86/02162.

A flow cell for the spectroscopic analysis of density gradients is described and shown in U.S. Pat. No. 3,728,032.

There is a need for an improved apparatus and method for detecting the presence of objects in a stream of liquid, in particular water, including sea water. It would be most advantageous if the apparatus and method could allow for the analysis of the liquid stream at high flow rates. For use in the analysis of water, in particular sea water, it would be most advantageous if the apparatus and method could be employed in a vessel, such as a ship, especially while the vessel is underway. The apparatus and method should preferably allow for the acquisition of data regarding the objects in the liquid stream, most preferably image data, for simultaneous or subsequent processing and analysis.

In a first aspect, the present invention provides a flow cell for the analysis of objects in a liquid stream, the flow cell comprising:
a flow cell body having a chamber therein defined by an inner surface of the flow cell body, the flow cell body having an inlet end and an outlet end;
the inlet end of the flow cell body being provided with a first fluid coupling member having a flow passage therethrough;
the outlet end of the flow cell body being provided with a second fluid coupling member having a flow passage therethrough;
the flow cell body comprising a first transparent portion, through which light may enter the chamber to illuminate objects within the chamber, and a second transparent portion through which objects within the chamber may be imaged;
wherein the chamber comprises a first transition portion adjacent the inlet end of the flow cell body, the first transition portion comprising a smooth transition between the flow passage of the first fluid coupling member and the inner surface of the flow cell body defining the chamber; and
wherein the chamber comprises a second transition portion adjacent the outlet end of the flow cell body, the second transition portion comprising a smooth transition between the flow passage of the second fluid coupling member and the inner surface of the flow cell body defining the chamber.

The flow cell of the present invention allows images of objects in a stream of fluid to be taken at high speed. Objects that may be imaged using the flow cell include, but are not limited to, organisms, for example plankton, bubbles and particles of matter.

In particular, the flow cell may be incorporated into an imager, as described in more detail hereinbelow.

The flow cell comprises a flow cell body. The flow cell body may be formed form any suitable material. In one preferred embodiment, the flow cell body is formed from plastic. Other suitable materials include metals, such as alloys, for example brass, steel, in particular stainless steel, and aluminium. In one preferred embodiment, the flow cell body is formed from brass, in particular an alpha beta or duplex brass, with Naval brass being especially preferred for many embodiments.

The flow cell body has an inlet end and an outlet end. In use, a liquid, such as water, flows through the flow cell body from the inlet end to the outlet end.

The inlet end is provided with a first fluid coupling member for connecting the flow cell body to a supply of the liquid to be analysed. The first fluid coupling comprises a flow passage therethrough for liquid. The first fluid coupling member may be of any suitable design. Suitable fluid couplings are known in the art. In many cases, the fluid coupling will comprise a male coupling member and a female coupling member. In such cases, the first fluid coupling member of the flow cell body is one of the male coupling member or the female coupling member, which may be connected to a corresponding female or male coupling member on a liquid supply line. In a preferred embodiment, the first fluid coupling at the inlet end of the flow cell body comprises a male coupling member. In this embodiment, the first fluid coupling is connectable to a corresponding female coupling member provided on a line or pipe through which the liquid being analysed is to be supplied. Couplings of this general configuration allow reliable and rapid water-tight coupling between inlet and outlet hoses or pipes and the inlet end and outlet end of the flow cell. This is in contrast with the use of other forms of coupling, such as simple clamps, for example jubilee clips or hose clips.

One preferred form of fluid coupling is a cam and groove coupling, also referred to in the art as a camlock coupling. The cam and groove coupling is known in the art and suitable cam and groove couplings are available commercially. An example of commercially available cam and groove couplings are the MIL C-27487 and EN 14420-7 specification couplings available from Trelleborg Industrie SAS.

Similarly, the outlet end of the flow cell body is provided with a second fluid coupling member for connecting the flow cell body to a line for removal of the liquid being analysed, once it has passed through the flow cell body. The second fluid coupling comprises a flow passage therethrough for liquid. The second fluid coupling member may be of any suitable design. Suitable fluid couplings are known in the art. In many cases, the fluid coupling will comprise a male coupling member and a female coupling member. In such cases, the second fluid coupling member of the flow cell body is one of the male coupling member or the female coupling member, which may be connected to a corresponding female or male coupling member on a liquid supply line. In a preferred embodiment, the second fluid coupling at the outlet end of the flow cell body comprises a male coupling member. In this embodiment, the second fluid coupling is connectable to a corresponding female coupling member provided on a line or pipe through which the liquid being analysed is to be removed.

As with the first fluid coupling at the inlet end, the preferred form of fluid coupling for the outlet end of the flow cell body is a cam and groove coupling, also referred to in the art as a camlock coupling. The cam and groove coupling is known in the art and suitable cam and groove couplings are available commercially. An example of commercially available cam and groove couplings are the MIL C-27487 specification couplings available from Trelleborg Industrie SAS.

The fluid coupling formed using the fluid coupling member on each of the inlet and outlet end of the flow cell body is preferably one that is reliable and easy to connect and disconnect. The fluid coupling should also provide a good alignment between the flow cell body and the conduit, line or pipe being connected to it at each end of the flow cell body.

The first and second fluid coupling members may be the same or different. For example, the first and second fluid coupling members may comprise parts of different types of coupling systems. Alternatively, the first and second coupling members may be different coupling members of the same coupling system, for example with one of the first and second coupling members being a male coupling member and the other being a corresponding female coupling member. Preferably, the first and second coupling members comprise parts of the same type of coupling system, especially a cam and groove coupling system. Preferably, the first and second coupling members are the same member, for example they are both either male coupling members or female coupling members.

The first and second fluid coupling members may be the same size or different sizes. In this respect, the size of the coupling members is a reference to the diameter of the flow passage through the coupling member. Preferably, the first and second coupling members are of the same size, that is have flow passages of the same diameter.

The flow cell body has a chamber therein defined by an inner surface of the flow cell body. The chamber is in flow communication with both the inlet and the outlet.

The flow cell body comprises a first transparent portion. The first transparent portion is disposed in the cell body between the inlet end and the outlet end. The first transparent portion extends through the wall of the cell body from the chamber within the cell body to the outer surface of the flow cell body. In this way, the first transparent portion acts as an optical window in the wall of the flow cell body and is disposed to allow light to enter the chamber from outside the flow cell body to illuminate objects within the liquid in the chamber.

The flow cell body further comprises a second transparent portion. The second transparent portion is disposed in the cell body between the inlet end and the outlet end. The second transparent portion extends through the wall of the cell body from the chamber within the cell body to the outer surface of the flow cell body. In this way, the second transparent portion acts as an optical window in the wall of the flow cell body and is disposed to allow objects within the chamber to be imaged from outside of the flow cell body using a suitable imaging apparatus.

The chamber within the flow cell body may be considered to have a detection zone, in which objects in the liquid in the detection zone may be both illuminated and imaged, with the first and second transparent portions arranged in the flow cell body to enable this illumination and imaging to take place. The detection zone may be considered to have a height, a width and a depth. The height of the detection zone is determined by the length of the region being imaged by the camera, for example, in the case of a line scan camera, the line length of the camera, as discussed in more detail below. The width of the detection zone is similarly determined by the width of the region being imaged by the camera, for example, in the case of a line scan camera, the number of lines, as also discussed in more detail below. The depth of the detection zone is determined by the width of the flow cell orthogonal to the direction of fluid flow through the detection zone, and relates to the depth of focus of the camera lens.

The flow cell body is at least partially transparent and comprises a transparent material to provide the first transparent portion and the second transparent portion, spaced apart from the first transparent portion. The first and second transparent portions of the flow cell body may be formed in any suitable manner. In particular, transparent material may be incorporated into the flow cell body to form the first and second transparent portions. In one preferred embodiment, the flow cell body is provided with a first opening for the first transparent portion and a second opening for the second transparent portion. A transparent material is mounted in each opening, to form the first and second transparent portions.

Alternatively, the flow cell body may be formed partly or wholly from a transparent material.

The first transparent portion and the second transparent portion are both disposed between the inlet end and the outlet end of the flow cell body, as mentioned above. The first transparent portion and the second transparent portion may be disposed at any position in the flow cell body, so as to allow objects in the liquid within the chamber to be both illuminated and imaged.

The first and second transparent portions of the flow cell body may be at any suitable position in the flow cell body and may be any suitable distance from the inlet end and the outlet end. The first and second transparent portions may be at the same position, that is distance, along the length of the flow cell body between the inlet end and the outlet end or may be in different positions along the length of the flow cell body between the inlet and outlet ends. Preferably, the first and second transparent portions are at the same position between the inlet end and the outlet end and are both the same distance from each of the inlet end and the outlet end of the flow cell body.

In one embodiment, the first transparent portion is equidistant from the inlet end and the outlet and is disposed centrally on the longitudinal axis of the flow cell body. Similarly, in one embodiment, the second transparent portion is equidistant from the inlet end and the outlet and is disposed centrally in the flow cell body. Preferably, this embodiment has both the first transparent portion and the second transparent portion equidistant from the inlet end and the outlet end of the flow cell body.

However, in an alternative embodiment the first transparent portion and/or the second transparent portion is closer to the outlet end of the flow cell body than the inlet end of the flow cell body. If this arrangement is employed, preferably both the first and second transparent portions are closer to the outlet end of the flow cell body than the inlet end of the flow cell body.

In particular, the positions of the first and second transparent portions and the detection zone of the chamber are preferably selected to minimise the occurrence of pressure waves within the detection zone. Pressure waves are created in the liquid stream flowing through the flow cell by discontinuities in the surfaces defining the flow passage through the flow cell. As described in more detail below, the form and shape of the surface defining the chamber within the flow cell has no discontinuities. However, discontinuities, such as step changes in the surface defining the flow passage, can occur upstream and/or downstream of the flow chamber and the detection zone thereof. For example, the couplings discussed hereinbefore may have discontinuities in their flow passages, in particular arising from a mismatch between the shape and size of the cross-section of the flow passage through the couplings member at the inlet and/or outlet end of the flow cell body and the pipe, conduit or line to which it is connected by the coupling. Such discontinuities give rise to pressure waves which move in the downstream direction from the inlet end of the flow cell body and/or which move in the upstream direction from the outlet end of the flow cell body. Pressure waves reaching the detection zone of the chamber within the flow cell body can adversely affect the imaging of objects in the fluid stream, for example increasing the extent of blurring of the images, deforming the images and/or losing detail from the images. For example, changes in the local velocity of the fluid arising from pressure waves propagating in the fluid can result in changes to the shape of the object being imaged or result in changes in the position of the object. If these shifts in the position of the object position are rapid, in relation to the camera sampling rate, and within the camera field of view, then the resultant shape of the object as seen in the image will be deformed or blurred.

The maximum size of discontinuity that can be accommodated is proportional to the velocity of the fluid travelling through the flow cell body. To minimise the formation of pressure waves within the chamber and the detection zone thereof, it is preferred that discontinuities in the surface defining the flow passage are below 1.5 mm, preferably below 1 mm, still more preferably below 0.5 mm.

As noted above, the detection zone of the chamber within the flow cell body is disposed at a position such that pressure waves generated as a result of discontinuities at the inlet end and/or outlet end of the flow cell body are at a minimum in the liquid in the detection zone. As will be appreciated, pressure waves generated at the inlet end of the flow cell body will propagate in the downstream direction further than pressure waves generated at the outlet end of the flow cell body, which must propagate in an upstream direction. To reduce or prevent pressure waves within the detection zone of the chamber within the flow cell body, it is preferred that the detection zone is disposed within the flow cell body such that the centre of the detection zone is at a distance of at least 70 mm from the inlet end of the cell body, more preferably at least 100 mm, still more preferably at least 130 mm, more preferably still at least 150 mm from the inlet end of the flow cell body. In one embodiment, the detection zone is about 170 mm from the inlet end of the cell body. In another embodiment, the centre of the detection zone is about 200 mm from the inlet end of the cell body. This distance ensures that pressure waves formed as a result of discontinuities in the surfaces defining the flow passage at the inlet end and/or its coupling to a liquid supply line do not propagate to the detection zone.

Similarly, it is preferred that the detection zone is disposed within the flow cell body at a distance of at least 50 mm from the outlet end of the flow cell body, more preferably at least 75 mm, still more preferably at least 100 mm, more preferably still at least 130 mm from the outlet end of the flow cell body. In one embodiment, the detection zone is about 170 mm from the outlet end of the cell body. In another embodiment, the centre of the detection zone is about 200 mm from the outlet end of the cell body. This distance ensures that pressure waves formed as a result of discontinuities in the surfaces defining the flow passage at the outlet end and/or its coupling to a line do not propagate upstream to the detection zone.

In some embodiments, the detection zone of the chamber within the flow cell body is preferably disposed such that the centre of the detection zone is closer to the outlet end than the inlet end of the flow cell body, more preferably such that the ratio of the distance of the detection zone from the inlet end to the distance of the detection zone from the outlet end is from 5:1 to 1:1, more preferably from 4:1 to 1:1, still more preferably from 3:1 to 1:1. However, in a preferred embodiment, the detection zone is located within the flow cell body such that the centre of the detection zone is equidistant from the inlet end and the outlet end.

In embodiments where the detection zone of the chamber is disposed centrally along the flow cell body, that is equidistant from both the inlet end and the outlet end of the flow cell body, the detection zone is spaced a sufficient distance from both ends to prevent pressure waves propagating from either end to the detection zone.

The first and second transparent portions may be arranged in the flow cell body at any suitable orientation to each other. In one preferred embodiment, the first transparent portion of the flow cell body is opposite the second transparent portion of the flow cell body, that is the first and second transparent portions are disposed symmetrically around the flow cell body. In this embodiment, the detection zone of the chamber is defined between the first and second transparent portions of the flow cell body and the first and second transparent portions lie on the optical axis, that is the line along which the imaging apparatus images the liquid within the detection zone. In this embodiment, it is particularly preferred that the centre of the first transparent portion and the centre of the second transparent portion lie on a line passing through and extending perpendicular to the central longitudinal axis of the flow cell body and the chamber therein.

Alternatively, the first and second transparent portions may be disposed asymmetrically around the flow cell body, such that the centres of the first and second transparent portions lie on a line that does not pass through the central longitudinal axis of the flow cell body. In such arrangements, the first transparent portion is not opposite the second transparent portion.

As noted above, the flow cell body has a chamber therein. The chamber is defined by an inner surface of the flow cell body. The chamber extends between the inlet end and the outlet end of the flow cell body and is in fluid communication with the passages in both of the first and second coupling members.

The chamber within the flow cell body may have any suitable form. Generally, the chamber is elongate, that is has a length between the inlet end and the outlet end of the flow cell body that is greater than the width or diameter of the chamber. As described hereinbefore, the chamber is of sufficient length to prevent pressure waves propagating through the liquid stream into the detection zone. As described hereinafter, the chamber is designed to allow the fluid flowing into the chamber from the inlet end to the detection zone to achieve a flow pattern that is as smooth as possible. Ideally, the flow pattern of the fluid flowing through the chamber is preferably substantially laminar flow. However, in practice, this may not be achievable, in which case the flow pattern is preferably transitional turbulent flow. In general, the requirement is to reduce the turbulence in the flow of fluid through the chamber, in order to allow objects to be imaged with minimal motion blurring, deformation or distortion of the image.

As described hereinafter, the shape and dimensions of the cross-section of the chamber within the flow cell body vary along the length of the flow cell body between the inlet end and the outlet end. Preferably, the cross-section of the detection zone of the chamber is of a constant shape and size along the length of the detection zone, that is from the inlet end of the detection zone to the outlet end of the detection zone. The detection zone of the chamber may have any suitable cross-sectional shape. Preferably, the detection zone of the chamber is rectangular in cross-section. A rectangular cross-section maximises the volume of fluid that can be imaged by the camera at any given point in time. This in turn maximises the volumetric flow rate of fluid through the detection zone and the flow cell body that can be achieved, while still maintaining the desired image quality.

As noted above, the detection zone within the chamber is considered to have a height, a width and a depth. The detection zone may have any suitable depth to allow objects in the liquid to be illuminated and imaged. In this respect, the term ‘depth’ as used in reference to the detection zone of the chamber in the flow cell is a reference to the depth of the detection zone of the chamber along the optical axis of the imaging apparatus, that is the line along which objects in the liquid within the detection zone are imaged by the imaging apparatus. The depth of the detection zone will, in many embodiments, be the distance between the first transparent portion and the second transparent portion, between which the detection zone of the chamber is defined. For the imaging of small objects, such as plankton or bubbles, it is typically the case that the optics of the imaging apparatus, in particular the imaging lens or lenses, have a very small depth of focus, which can be as little as 0.5 mm. However, in order to achieve a sufficient volumetric flow rate of liquid through the flow cell, the detection zone of the chamber has a depth that is greater than the depth of focus of the imaging apparatus. This will result in at least some of the objects in the liquid within the chamber being out of sharp focus or blurred when imaged. The amount of blurring of the objects that is acceptable will depend upon the image classification technology employed to classify and interpret the image data. Suitable image classification technology are known in the art and their ability to handle blurred image data will be known and understood.

In general, the detection zone of the chamber may have a depth that is at least 2 times the depth of field of the imaging apparatus, preferably at least 3 times, more preferably at least 4 times, still more preferably at least 5 times, more preferably still at least 6 times, especially at least 7 times the depth of field of the imaging apparatus being employed with the flow cell. The detection zone of the chamber may have a depth that is up to 40 times the depth of field of the imaging apparatus or even larger, especially in embodiments in which the imaging apparatus has a very small depth of field. The detection zone preferably has a depth up to 35 times the depth of field of the imaging apparatus, more preferably up to 30 times, still more preferably up to 25 times, more preferably still up to 20 times, especially up 15 times the depth of field of the imaging apparatus being employed with the flow cell. The detection zone of the chamber may have a depth that is from 2 to 40 times the depth of field of the imaging apparatus, preferably from 3 to 35 times, more preferably from 4 to 30 times, still more preferably from 5 to 25 times, more preferably still from 6 to 20 times, especially from 7 to 15 times the depth of field of the imaging apparatus being employed with the flow cell.

For example, in the case of an imaging apparatus with optics having a depth of field of 0.5 mm, the depth of the detection zone of the chamber may be from 10 to 20 mm, more preferably from 11 to 18 mm, still more preferably from 12 to 16 mm, more preferably still from 13 to 15 mm, for example about 14 mm.

The depth of the detection zone, by being significantly greater than the depth of field of the imaging apparatus, provides a compromise between having all objects sharply focussed in the images and ensuring a sufficient volumetric flow rate of liquid through the detection zone. Selection of the dimensions of the depth of the detection zone, as discussed above, allows objects to be imaged with sufficient sharpness of detail to allow the images to be processed and the objects identified or otherwise processed, while at the same time providing a sufficient volumetric flow rate of liquid through the chamber to allow the sampling and imaging procedure to be carried out sufficiently rapidly to be practicable on a commercial or large scale.

As noted above, the detection zone within the chamber is considered to have a height, that is the width of the chamber along a first line perpendicular to the optical axis. The height of the detection zone perpendicular to the optical axis will, in many embodiments, be the distance between the walls of the flow cell body defining the detection zone orthogonal to the optical axis and the line extending between the first transparent portion and the second transparent portion, between which the detection zone of the chamber is defined. The detection zone of the chamber may have any suitable height perpendicular to the optical axis. The height perpendicular to the optical axis may be selected according to the properties of the imaging apparatus being employed to image objects within the detection zone. In particular, the width perpendicular to the optical axis may be selected to maximise the volume of the detection zone that is imaged. The maximum height perpendicular to the optical axis is dependent upon the properties of the imaging apparatus. For example, the maximum height may be determined by the field of view of the imaging apparatus. By having the height of the detection zone perpendicular to the optical axis at or close to the width of the field of view of the imaging apparatus, all or substantially all of the detection zone may be imaged. Preferably, the height of the detection zone perpendicular to the optical axis is at least 75% of the width of the field of view of the imaging apparatus, more preferably at least 80%, still more preferably at least 85%, more preferably still at least 90%, still more preferably at least 95%, especially at least 97% of the width of the field of view of the imaging apparatus.

Any suitable imaging apparatus may be employed with the flow cell of the present invention. Suitable cameras for taking images are known in the art and are commercially available. Suitable cameras include area-scan cameras and line scan cameras. In one preferred embodiment, the imaging apparatus comprises a line scan camera. Line scan cameras have the advantageous property of sampling the field of view continuously at high resolution, and as such can ensure that all the fluid passing in front of the camera will be imaged to that resolution. Area-scan cameras cannot ensure such imaging and require significant post-processing of sequential images to ensure no over sampling of the fluid flow. Suitable line scan cameras are known in the art and are commercially available.

The line scan camera has an array of pixels arranged in one or more lines, each line capturing a line of image at each scan. The line scan camera may capture one line at a time, that is a monochrome camera. Alternatively, the camera may capture two lines at a time, for example a Bayer encoded colour camera; three lines at a time, one each of red, blue and green pixels or similar encoded colour camera; or more than three lines, as in the case of a time domain integration (TDI) camera. A TDI camera can sample more than one line of pixels in each scan and, over time, repeated sampling is integrated to produce a final image line. In this way, as an object, suspended in a moving fluid, moves through the imaging field of view of the camera, its image is successively captured by lines of pixels that are added together, that is integrated, into one final line of pixels, when is then output from the camera to the connected computer.

In one embodiment, the line scan camera has an array of 3 lines of pixels, one line each for red, green and blue pixels, each line comprising 8,192 pixels. The pixels are 5 μm square and occupy 15 μm by 40.96 mm at the focal plane of the optics.

Alternatively, line scan cameras having 3 lines each of 16,384 pixels or 64 lines each of 16,384 pixels can also be used.

One suitable range of line scan cameras are the Linea ML and HS cameras of Teledyne Dalsa, Canada.

As noted above, the detection zone is considered to have a width. The width of the detection zone is along a second line perpendicular to the optical axis of the imaging apparatus and is parallel to the axis of flow of fluid through the detection zone. Preferably, the width of the detection zone perpendicular to the optical axis is selected according to the length of the active line scanned by the sensor pixels of the line scan camera. In particular, it is preferred that the width of the detection zone perpendicular to the optical axis is no greater than the length of the active line of the line scan camera, more preferably slightly shorter than the length of the active line of the line scan camera. In this way, it is assured that the entire width of the detection zone is imaged and, more particularly, that all objects passing through the detection zone are imaged. Preferably, the width of the detection zone perpendicular to the optical axis is at least 75% of the length of the active line of the line scan camera, more preferably at least 80%, still more preferably at least 85%, more preferably still at least 90%, still more preferably at least 95%, especially at least 99.6% of the length of the active line scanned by the sensor pixels of the line scan camera.

The imaging apparatus may comprise, in addition to the camera, a suitable optical assembly, for example an array of one or more lenses. Suitable lenses for use in the optical assembly to allow images of the objects in the liquid in the detection zone of the chamber to be captured by the camera are known in the art. In particular, suitable macro lenses are known. However, it has been found that using standard macro lenses, that is entocentric lenses, results in a trapezoidal view field, that effectively creates a shoulder of shadowing appearing at the edges of the image of the rectangular cross-section. Preferably, therefore, the optical assembly comprises a telecentric lens. This ensures that the internal edges of the chamber within the flow cell body are imaged, without any shadowing. The telecentric lens will also ensure that the magnification of all objects within the detection zone is the same, that is ensures magnification of 1.0 for all objects. This in turn ensures that the scale and dimensions of the objects appearing in the images can be readily determined. In the case of imaging plankton for identification, the size of the imaged object can be important in identifying plankton and determining the species.

The first transparent portion of the flow cell body may be any suitable shape and size to allow for adequate illumination of the liquid within the detection zone by a light source positioned outside the flow cell body. The first transparent portion may be considered to have a height and a width. The height of the first transparent portion extends perpendicular to the general direction of fluid flow through the detection zone. The width of the first transparent portion extends parallel to the general direction of fluid flow through the detection zone. To ensure that the liquid within the detection zone is sufficiently illuminated, it is preferred that the height of the first transparent portion is at least the same as the height of the detection zone.

The second transparent portion of the flow cell body may be any suitable shape and size to allow for the liquid within the detection zone to be imaged through the second transparent portion by an imaging apparatus positioned outside the flow cell body. The second transparent portion may be considered to have a height and a width. The height of the first transparent portion extends perpendicular to the general direction of fluid flow through the detection zone. The width of the first transparent portion extends parallel to the general direction of fluid flow through the detection zone. To ensure that the liquid within the detection zone is imaged properly, it is preferred that the height of the second transparent portion is at least the same as the height of the detection zone.

The first and second transparent portions may have any suitable shape. They may be the same shape or different shapes, more preferably the same shape, for ease of construction of the flow cell. Circular is one preferred shape for one or both of the transparent portions.

In general, the shape of the cross-section of the flow passage through a fluid coupling, such as a cam and groove coupling, is generally circular. In the majority of embodiments of the flow cell of the present invention, the cross-section of the detection zone of the chamber will have a different shape and a different cross-sectional area to the flow passage through the fluid coupling. In particular, as noted above, in one preferred embodiment the cross-section of the detection zone of the chamber is rectangular. As discussed above, it is necessary to avoid the formation of pressure waves which may propagate either upstream or downstream to the detection zone within the chamber. It is also necessary that the liquid flow stream passing through the detection zone is as smooth as possible and that turbulence within the liquid stream in the region of the detection zone is minimised.

To this end, the chamber within the flow cell body comprises a first transition portion adjacent the inlet end of the flow cell body. The first transition portion comprises a smooth transition between the flow passage of the first fluid coupling member and the inner surface of the flow cell body defining the chamber. Similarly, the chamber comprises a second transition portion adjacent the outlet end of the flow cell body. The second transition portion comprises a smooth transition between the flow passage of the second fluid coupling member and the inner surface of the flow cell body defining the chamber.

The first and second transition portions each provide a smooth transition in the shape of the cross-section of the chamber from the shape of the cross-section of the flow passage through the respective first and second couplings to the shape of the cross-section of the detection zone within the chamber. The surface of one or both transition portions may be multifaceted, that is the surface may comprise a plurality of longitudinal and/or circumferential facets or portions. More preferably, the surface of one or both, most preferably both, transition portions has single facet in both the longitudinal and circumferential directions. This form of surface provides for the smoothest and least turbulent flow pattern of liquid between the inlet end of the flow cell body and the detection zone and between the detection zone and the outlet end of the flow cell body.

Each of the first and second transition portions may have any suitable form for its surface that provides a smooth transition from the cross-sectional shape of the inlet and the outlet ends of the flow cell body to the cross-sectional shape of the chamber and the detection zone therein. The forms of the first and second transition portions may the same or different. In one preferred embodiment, the surface defining the first transition portion has the same form as the surface defining the second transition portion.

Suitable curve functions for defining the form of the surface defining the first and second transition portions are known in the art. Suitable smoothed curve functions, more generally polynomial basis functions include Bezier, Legendre, Chebyshev or Bernstein polynomial functions. In one preferred embodiment, the surface of the first transition portion and/or the second transition portion are defined by a set of Bezier curves.

As discussed above, the first transition portion extends from the inlet end of the flow cell body in the downstream direction towards the detection zone within the chamber. The first transition portion preferably extends at least 50% of the distance between the inlet end of the flow cell body and the inlet end of the detection zone, more preferably at least 60%, still more preferably at least 70%, more preferably still at least 80%, especially at least 85% of the distance between the inlet end of the flow cell body and the inlet end of the detection zone. The first transition portion may extend to the inlet end of the detection zone. However, it is preferred that the first transition portion ends at a position upstream of the detection zone. Preferably, the first transition portion extends up to 95% of the distance between the inlet end of the flow cell body and the inlet end of the detection zone, more preferably up to 90%. In one preferred embodiment, the first transition portion extends from the inlet end of the flow cell body about 87.5% of the distance from the inlet end of the flow cell body to the inlet end of the detection zone.

Similarly, as discussed above, the second transition portion extends from the outlet end of the flow cell body in the upstream direction towards the detection zone within the chamber. The second transition portion preferably extends at least 50% of the distance between the outlet end of the flow cell body and the outlet end of the detection zone, more preferably at least 60%, still more preferably at least 70%, more preferably still at least 80%, especially at least 85% of the distance between the outlet end of the flow cell body and the outlet end of the detection zone. The second transition portion may extend to the outlet end of the detection zone. However, it is preferred that the second transition portion ends at a position downstream of the detection zone. Preferably, the second transition portion extends up to 95% of the distance between the outlet end of the flow cell body and the outlet end of the detection zone, more preferably up to 90%. In one preferred embodiment, the second transition portion extends from the outlet end of the flow cell body about 87.5% of the distance from the outlet end of the flow cell body to the outlet end of the detection zone.

As discussed above, the flow cell of the present invention finds use in an imaging assembly for imaging objects in a liquid stream.

Accordingly, in a further aspect, the present invention provides an imaging assembly for imaging objects in a liquid stream, the imaging assembly comprising:
a flow cell as hereinbefore described;
an imaging apparatus for imaging objects in the liquid within the detection zone of the flow cell through the second transparent portion of the flow cell body; and
an illumination apparatus for generating light to illuminate a liquid in the detection zone of the flow cell through the first transparent portion of the flow cell body.

The imaging assembly of this invention may be used to image a wide range of objects within a liquid stream. In one preferred embodiment, the imaging assembly is used to image plankton and detritus in sea water or fresh water passed through the flow cell. In another preferred embodiment, the imaging assembly is used to image bubbles of gas in water, for example sea water, passed through the flow cell.

The imaging assembly comprises an imaging apparatus. The imaging apparatus comprises a camera for capturing an image of objects in the liquid in the detection zone of the flow cell through the second transparent portion of the flow cell body. The imaging apparatus preferably further comprises an optical assembly comprising one or more lenses disposed between the camera and the flow cell body. Details of the imaging apparatus and the optical assembly are as described above.

The imaging assembly further comprises an illumination apparatus. The illumination apparatus provides light to the liquid within the detection zone through the first transparent portion of the flow cell body. The illumination apparatus comprises a light source. Any suitable means for generating a source of light may be used. In general, the light source provides light to illuminate objects within the detection zone at a sufficient intensity to allow the objects to be imaged by the imaging apparatus at the camera shutter speed required to capture an image of the objects at the speed they are passing through the detection zone. In particular, faster moving objects, due to higher liquid velocities, require a faster shutter speed to capture an image that is sufficiently clear, which in turn requires light of a higher intensity to perform the imaging adequately.

Suitable illumination sources are known in the art, for example Xenon arc lamps, multispectral lasers and light emitting diodes.

Preferably, the light source is a light emitting diode (LED). Suitable LEDs are known in the art and are commercially available and are more compact and robust than Xenon lamps and lasers.

In general, smaller LED sources behave more closely as point light sources. The light from such smaller LEDs is therefore easer to focus into a collimated beam with very low beam divergence. However, the intensity of the light emitted by such LEDs is low, which adversely affects the imaging of small objects, such as plankton, detritus and bubbles in a liquid stream. It is therefore preferred to use an LED having a higher luminous flux. In particular, the LED preferably has a luminous flux of from 50 cd/mm², more preferably from 100, still more preferably from 150, more preferably still at least 200, especially at least 250 cd/mm². The LED preferably has a luminous flux of from 100 to 250 cd/mm², more preferably from 150 to 225, still more preferably from 175 to 215 cd/mm². An LED having a luminous flux of about 200 cd/mm² is preferred for many embodiments. LEDs having a higher luminous flux may also be employed with good results. In general, it is preferred to use LEDs with as high a luminous flux as possible.

In general, the higher brightness of the LED allows the camera to be used at its fastest operating setting. In the case of a line scan camera, the higher brightness LEDs allow a setting of 100K lines per second to be used, with an exposure of 4 microseconds or less. This ensures a sharp image with a low amount of blurring arising from motion of the object being imaged.

It is known in imaging plankton to use red light (having a wavelength of 680 nm for example), since algae cannot use red light to photosynthesise and hence, they will not grow on the illuminated regions of a flow cell. However, in the present invention it is preferred to use white light to illuminate objects in the flow cell. Bacteria will attach to all available surfaces within the flow cell, which will necessitate regular cleaning, regardless of light spectral content. White light allows the colourations of many plankton to be detected, which assists in their identification. The colourations are generated by broad-spectrum phosphors being excited by irradiation by short wavelength blue light (having a wavelength of approximately 430 nm) within the LED structure.

A cooling system may be required to remove heat generated by the LED when in operation.

The illumination apparatus preferably further comprises an optical assembly. The function of the optical assembly is to collimate the light generated by the light source. The optical assembly may comprise one or more lenses to collimate the light. Suitable lenses for use in the optical assembly are known in the art. Preferably, the optical assembly comprises a telecentric lens. This allows reduces or eliminates constraints on the position of the flow cell in relation to the light source. A telecentric lens also reduces scattering of light.

In a preferred embodiment, the optical assembly comprises an achromatic telecentric lens. In this way, colour aberrations in the collimated beam of light are reduced or completely avoided.

In a particularly preferred embodiment, the light emitted by the light source is caused to pass through a pin hole before entering the optical assembly. The use of a pin hole reduces the emission area of the light beam. Without the use of a pin hole, the larger area would cause a decrease in the edge contrast of objects being imaged.

The light emitted by the LED is incoherent. The light passing through the pin hole is partially collimated. That is, the beam of light leaving the pin hole is less divergent than the incident beam of light. The pin hole is employed to generate a beam of light that is within the acceptance angle of a collimating lens, typically about 14°.

The optimum size for the pin hole is a diameter of 0.458 mm, to provide a light beam with the most preferred properties for imaging objects. However, the use of a pin hole in this way can significantly reduce the intensity of the light. However, the extent to which the pin hole collimates the light is reduced as the diameter of the pin hole increases. Accordingly, it may be preferred to use a pin hole with a larger diameter, as a compromise between the optimum optical performance and increased light intensity. Therefore, it is preferred to use a pin hole having a diameter that is at least 110% of the theoretical optimum size, preferably at least 120%, more preferably at least 130%, still more preferably at least 140%, more preferably still at least 150%, especially at least 160%, more preferably at least 170%. A pin hole having a diameter up to 270% of the optimum may be used, preferably up to 260%, more preferably up to 250%, still more preferably up to 240%, more preferably still up to 230%, especially up to 225%. A pin hole having a diameter of from 150 to 250% of the theoretical optimum may be used, preferably from 160 to 240%, more preferably from 170 to 230%, especially from 175 to 220%. A pin hole of 0.5 mm or greater may be employed, for example a pin hole of at least 0.5, 0.55, 0.6, 0.65, 0.7, 0.75, 0.8, 0.85, 0.9, 0.95 or 1.0 mm. Pin holes of larger diameter may be also be used, such as a diameter of 1.05, 1.1, 1.15, 1.2, 1.25, 1.3, 1.35, 1.4, 1.45 or 1.5 mm. A pin hole in the range of from 0.5 to 1.5 mm may be used, preferably from 0.6 to 1.4 mm, more preferably from 0.65 to 1.3 mm, still more preferably from 0.7 to 1.2 mm, more preferably still from 0.75 to 1.1 mm, especially from 0.8 to 1.0 mm.

Using the pin hole of the aforementioned diameter, the light leaving the pin hole is less divergent than the incident beam of light, that is the rays of light are very close to parallel, with the angle of the light beam being less than 10°, preferably less than 8°, more preferably less than 7°, still more preferably less than 6°, especially about 5°. In many embodiments, a divergence of 2° or less in the light leaving the pin hole is achieved.

The light beam leaving the pin hole is slightly diverging, as noted above. This beam of light is passed through the optical assembly comprising a telecentric lens, as also noted above. In one preferred arrangement, the distance between the telecentric lens and the pin hole is such that the diameter of the beam of light incident on the telecentric lens is the same as the diameter of the lens.

The pin hole is preferably placed at the focal point of the telecentric lens. This minimises non-parallel rays of light emitted by the LED reaching the lens.

It has been found that, even when using a pin hole with a diameter larger than the theoretical optimum diameter, the quality of the image of objects in the liquid stream obtained is significantly better than images obtained in the same arrangement without a pin hole.

Accordingly, in a further aspect, the present invention provides an apparatus for illuminating objects, the apparatus comprising:
a light source for generating light;
a first collimator for collimating the light emitted by the light source, the first collimator comprising a pin hole through which the light from the light source is caused to pass; and
a second collimator comprising a telecentric lens.

As noted above, the present invention provides an imaging assembly for imaging objects in a liquid stream. The imaging assembly comprises a flow cell as hereinbefore described; an imaging apparatus for imaging objects in the liquid within the detection zone of the flow cell; and an illumination apparatus for generating light to illuminate a liquid in the detection zone of the flow cell.

It is preferred to provide the assembly with a housing to surround at least the imaging apparatus. In general, the imaging apparatus should be prevented from coming into contact with dust and fluids, in particular water vapour. The housing is preferably constructed to have an IP67 rating. The housing may be formed from any suitable material. In one preferred embodiment, the housing is formed from a plastic, in particular acrylic.

It is preferred to provide the housing to enclose both the imaging apparatus and the illumination apparatus. Again, this serves to protect the imaging and illumination apparatus from the ingress of dust and fluids.

In a preferred embodiment, the assembly is provided with an environment control system for controlling the environment within the housing. The environment control system operates to control the temperature and humidity within the housing. This is particularly preferred, in order to provide the optimum environment for the optical assembly of the imaging apparatus. The environment control system also accommodates the heat generated by the imaging apparatus, in particular the camera, and the light source, to avoid excessively high temperatures within the housing. Suitable environment control systems are known in the art. In one preferred embodiment, the environment control system is a Peltier-effect control system.

In a further aspect, the present invention provides a method of generating a collimated beam of light, the method comprising:
generating a first beam of light, the first beam being a beam of divergent, incoherent light;
collimating the first beam of light, collimating comprising:
   in a first step passing the first beam of light through a pin hole to produce a second beam of light less divergent that the first beam of light; and
   in a second step passing the second beam of light through a collimator comprising a telecentric lens.

In a still further aspect, the present invention provides a method for imaging objects in a liquid, the method comprising:
feeding the liquid to the inlet end of a flow cell as hereinbefore described;
generating a beam of light;
collimating the beam of light to form a collimated beam of light;
passing the collimated beam of light through the first transparent portion of the flow cell body of the flow cell; and
capturing an image of objects within the liquid in the flow cell body through the second transparent portion of the flow cell body.

As discussed above, the method of the present invention finds particular use in the imaging of objects in water, in particular in sea water from estuaries, seas or oceans. The method is particularly suitable for imaging plankton, detritus and bubbles in the water.

Embodiments of the present invention will now be described, by way of example only, having reference to the accompanying drawings, in which:

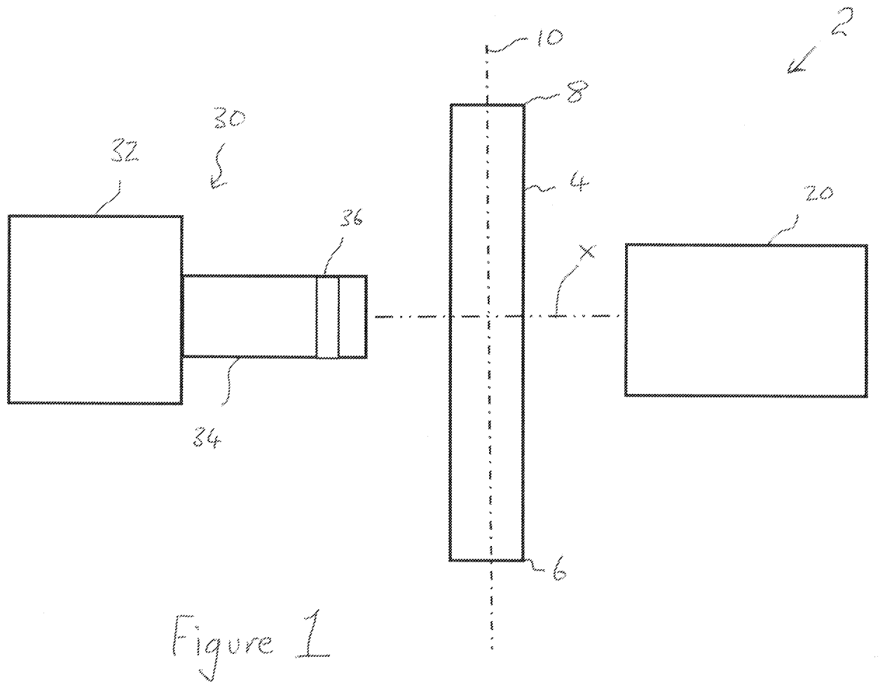

FIG. 1 is a diagrammatic representation of an imaging assembly for imaging objects in a liquid stream according to one embodiment of the present invention;

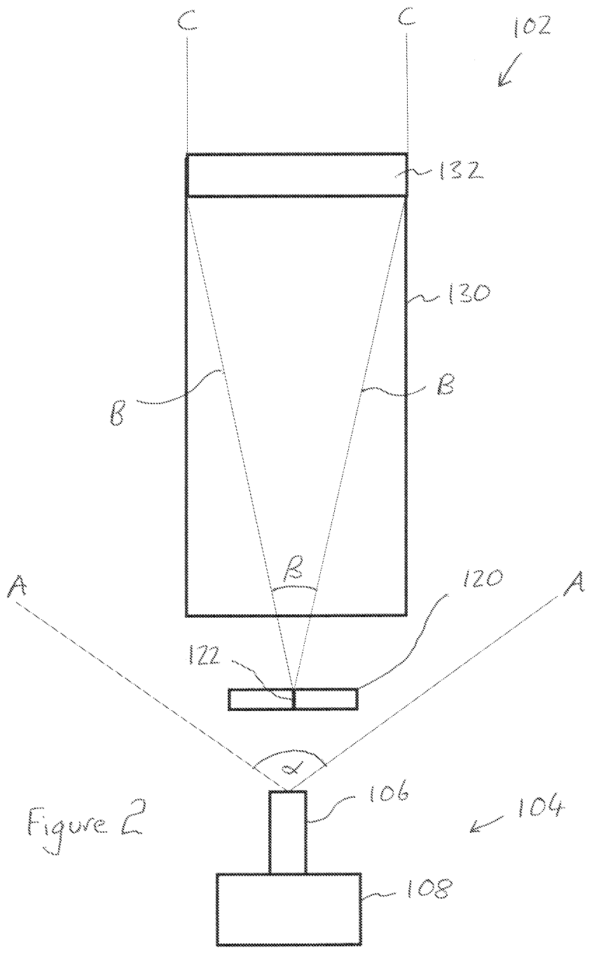

FIG. 2 is a diagrammatic representation of an apparatus for illuminating objects according to one embodiment of the present invention;

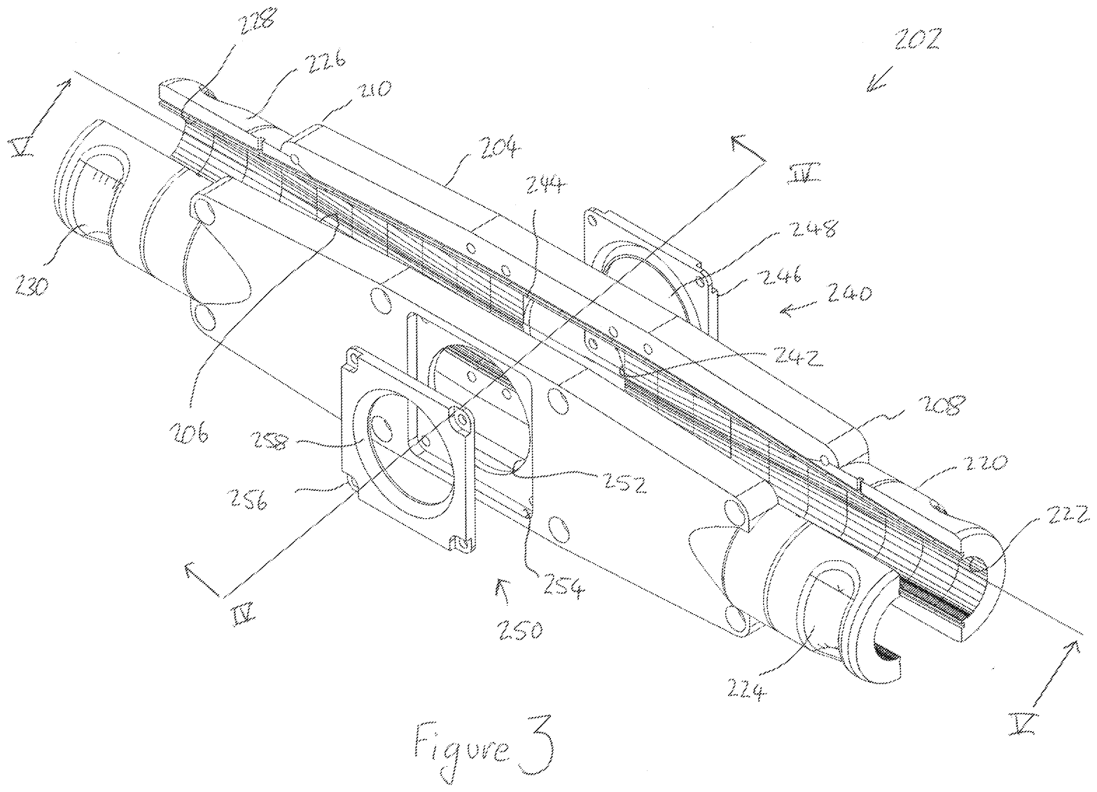

FIG. 3 is a perspective exploded view of one embodiment of the flow cell of the present invention;

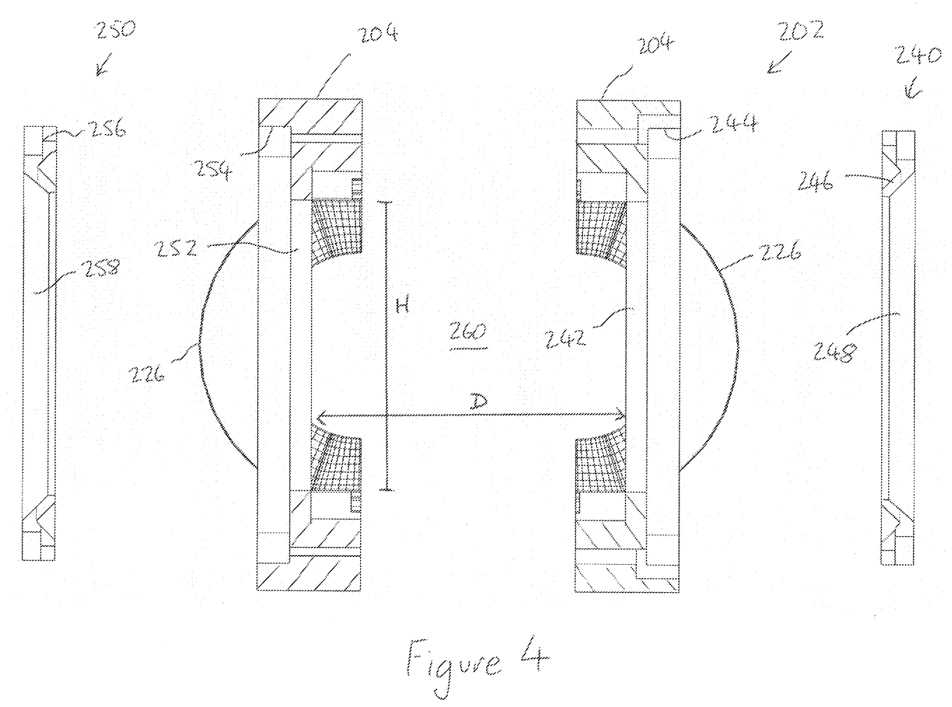

FIG. 4 is a lateral cross-sectional view of the flow cell of FIG. 3 along the line IV-IV;

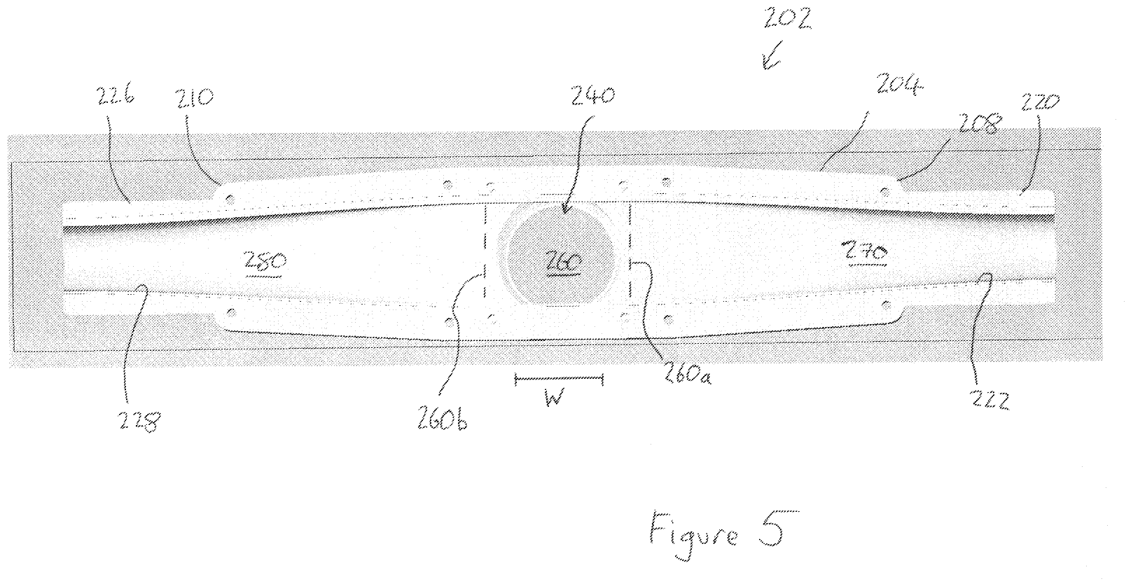

FIG. 5 is a longitudinal cross-sectional view of the flow cell of FIG. 3 along the line V-V;

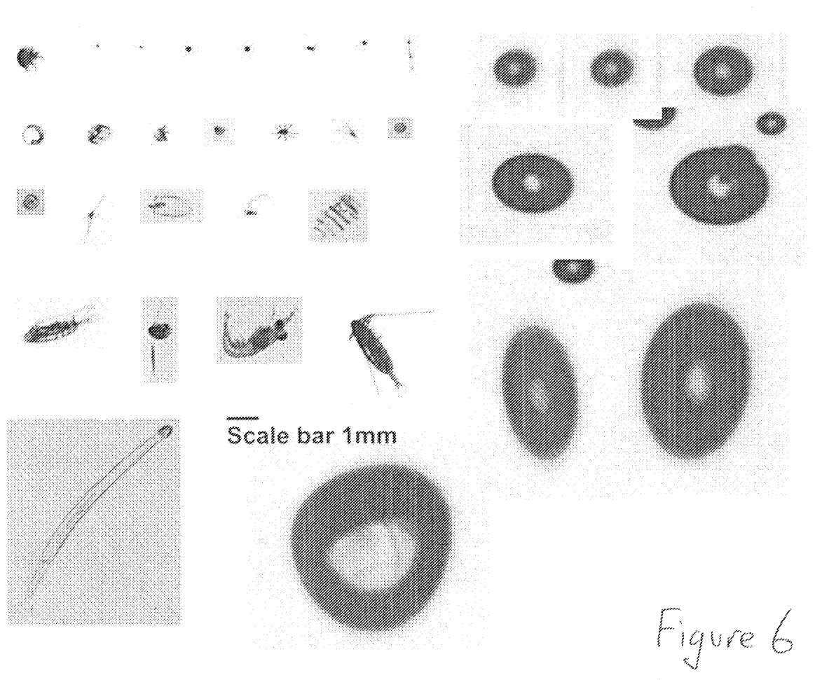

FIG. 6 shows two sets of images of objects taken using the apparatus of FIGS. 1 to 5; and

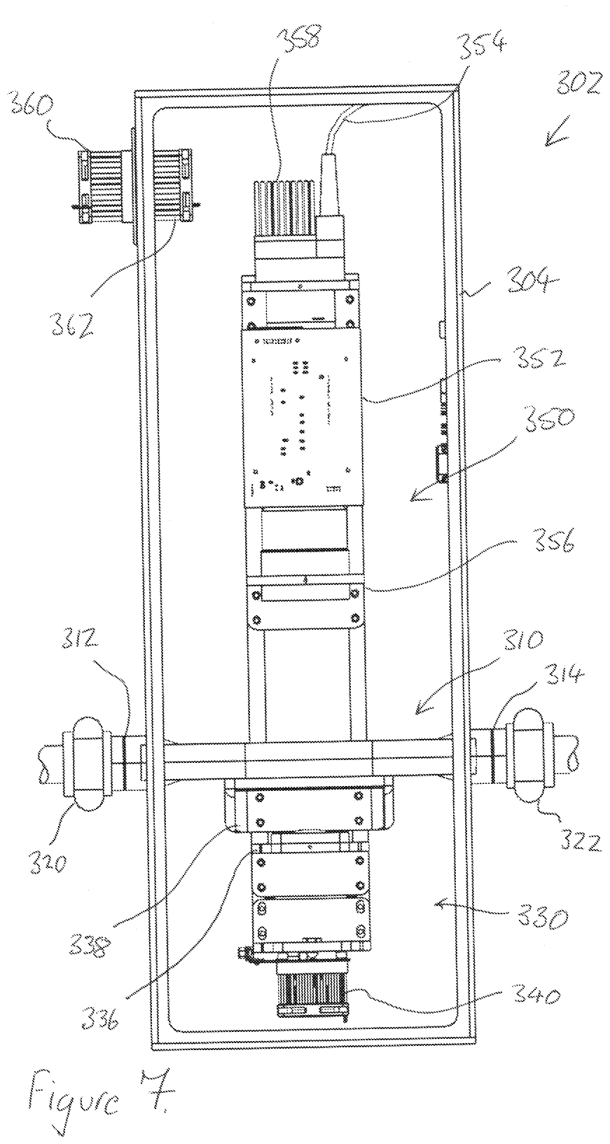

FIG. 7 is a representation of an imaging assembly for imaging objects in a liquid stream according to a further embodiment of the present invention.

Turning to FIG. 1, there is shown an imaging assembly for imaging objects in a liquid stream according to one embodiment of the present invention. The imaging assembly, generally indicated as 2, comprises a flow cell 4 having an inlet end 6 and an outlet end 8. The flow cell 4 has a central longitudinal axis 10. In use, a liquid stream having objects to be imaged is introduced into the flow cell 4 through the inlet end 6 and leaves the flow cell through the outlet end 8.

The imaging assembly 2 further comprises an illuminating apparatus 20 for illuminating objects. In use, the illuminating apparatus 20 provides a source of collimated light for illuminating objects entrained in the liquid stream within the flow cell 4.

The imaging assembly 2 further comprises an imaging apparatus, generally indicated as 30, comprising a line scan camera 32. The camera has an array of 3 lines each of 8,192 pixels, that is a full-colour camera, with a line for each red, green and blue pixels. The pixels are each 5 μm square, and hence occupy 15 μm by 40.96 mm at the focal plane of the optics.

The imaging apparatus 30 further comprises an optical assembly 34. The optical assembly 34 includes one or more lenses. In particular, the optical assembly 34 comprises a telecentric lens 36. The distance of the imaging apparatus 32 from the longitudinal axis 10 of the flow cell 4 is determined by the properties of the optical assembly 34. In the embodiment represented in FIG. 1, the optical assembly 34 is about 130 mm from the longitudinal axis 10 of the flow cell 4.

The imaging assembly 2 has an optical axis X, that is the centre line along which the imaging apparatus takes images of objects in the flow cell 4, which extends perpendicular to the longitudinal axis 10.

Turning to FIG. 2, there is shown a diagrammatic representation of an illuminating apparatus for illuminating objects according to one embodiment of the present invention. The illuminating apparatus, generally indicated as 102, finds general use in the illumination of objects, but is particularly suitable for use in the imaging assembly 2 of FIG. 1.

The illuminating apparatus 102 comprises a light source 104 comprising a light emitting diode (LED) 106. A cooling system 108 is provided to remove heat generated by the LED during operation. The LED is a high output variety, for example OSRAM KW CSLPM1.TG. The LED has an emitting area of 1.25 mm×1.59 mm with a luminous flux of around 200 cd/mm² from a single silicon diode.

In use, the LED emits a field of light indicated by the lines A. The field of light A is generally a broad field, with rays of light extending radially from the LED. For example, the field of light A may have light rays extending through an angle α, for example 120°.

Light from the LED 106 is directed to a plate 120 having a pin hole 122 therein. Light rays pass through the pin hole 122. The light leaving the LED is divergent. The light leaving the pin hole 122 is less divergent. The light rays leaving the pin hole 122 are indicated by the lines B in FIG. 2 and are substantially parallel, having a field angle of β, typically about +/−5°, depending upon the diameter of the pin hole. One suitable size for the pin hole 122 which provides a sufficient collimation of the incident light together with sufficient light intensity to illuminate objects within the flow cell is from 0.8 to 1 mm.

The beam of light B leaving the pin hole 122 is passed through a collimator 130 comprising an achromatic telecentric lens 132. The telecentric lens 132 is positioned relative to the pin hole 122 such that the diameter of the light beam B is substantially the same as the diameter of the telecentric lens 132 at the focal point of the lens. The light passing through the collimator 130 is collimated, with the beam of light C leaving the collimator 130 having rays very close to parallel, that is a field angle of less than +/−2°.

The beam of light C may be used to illuminate a wide range of objects and objects. In particular the beam of light C may be directed at the flow cell 4 in the imaging assembly of FIG. 1, described above.

Turning to FIG. 3, there is shown an exploded perspective view of one embodiment of the flow cell according to the present invention. The flow cell, generally indicated as 202, comprises a flow cell body 204. The flow cell body 204 is elongate and is generally rectangular in cross-section. The flow cell body 204 has an elongate chamber 206 therein. The chamber 206 has a central portion having a rectangular cross-section. In the embodiment shown in the figures, the flow cell body 204 is formed from Naval brass in two longitudinal halves, which are secured together by screws and made watertight using a gasket or an o-ring (not shown for clarity).

The flow cell body 204 has an inlet end 208 and an outlet end 210. The inlet end 208 is provided with a first fluid coupling member 220 having a flow passage 222 therethrough. The flow passage 222 is circular in cross-section. The coupling member 220 is the male component of a cam and groove coupling system. The coupling member 220 is generally tubular and comprises a transverse groove 224 formed in its outer surface. Similarly, the outlet end 210 is provided with a second fluid coupling member 226 having a flow passage 228 therethrough. The flow passage 228 is circular in cross-section. The coupling member 226 is the male component of a cam and groove coupling system. The coupling member 226 is generally tubular and comprises a transverse groove 230 formed in its outer surface.

The flow cell body 204 is provided with a first transparent portion 240, through which light may enter the chamber 206 to illuminate objects within the chamber. To provide the first transparent portion 240, the flow cell body 204 has a circular opening 242 formed therein. The opening 242 lies within a square recess 244 formed in the outer surface of the flow cell body 204. A square frame member 246 holds a circular transparent window 248 and is mounted in the square recess 244 by screws (not shown for clarity).

The flow cell body 204 is further provided with a second transparent portion 250, through which images of objects within the chamber 206 may be taken. The second transparent portion 250 is located opposite the first transparent portion 240. To provide the second transparent portion 250, the flow cell body 204 has a circular opening 252 formed therein. In the same manner as for the first transparent opening 240, the opening 252 lies within a square recess 254 formed in the outer surface of the flow cell body 204. A square frame member 256 holds a circular transparent window 258 and is mounted in the square recess 254 by screws (not shown for clarity).

The arrangement of the first and second transparent portions 240, 250 is also shown in cross-section in FIG. 4. As can be seen, the first and second transparent portions 240, 250 are disposed opposite one another and are centrally located between the inlet end 208 and the outlet end 210 of the flow cell body. A detection zone, indicated as 260, is defined in the region of the chamber between the first and second transparent portions 240, 250. The detection zone 260 has an inlet end 260a and an outlet end 260b.

The detection zone 260 has a depth D, a height H and a width W, as indicated in FIGS. 4 and 5.

The depth D of the detection zone 260 between the first and second transparent portions 240, 250, that is along the optical axis X, is 13.8 mm. The depth D of the detection zone 260 in across the chamber 206 perpendicular to the optical axis X is 40.8 mm.

The height H and width W of the detection zone are defined by the diameter of the transparent windows 248, 258. The diameter of the transparent windows 248, 258 is the same and is selected to be slightly less than the length of the scan line of the line scan camera 32. In this way, the line scan camera will detect and image the edges of the transparent window, allowing imaging of the full height of the fluid passing through the flow cell.

The chamber 206 is shown in longitudinal cross-section in FIG. 5. The chamber 206 comprises a first transition portion 270 adjacent the inlet end 208 of the flow cell body 204. The first transition portion 270 has an inner surface of the flow cell body 204 that provides a smooth transition between the circular flow passage 222 of the first fluid coupling member 220 and the inner surface of the flow cell body defining the rectangular chamber 206. In particular, the surface of the transition portion 270 may be defined by a set of Bezier curves that achieve the smooth transition from a passage having a circular cross-section and the rectangular cross-section of the chamber 206.

The chamber 206 further comprises a second transition portion 280 adjacent the inlet end 210 of the flow cell body 204. The second transition portion 280 has an inner surface of the flow cell body 204 that provides a smooth transition between the circular flow passage 228 of the second fluid coupling member 228 and the inner surface of the flow cell body defining the rectangular chamber 206. In particular, the surface of the transition portion 280 may be defined by a set of Bezier curves that achieve the smooth transition from a passage having a circular cross-section and the rectangular cross-section of the chamber 206.

As can be seen in FIG. 5, the inner surface of the flow cell body 204 defining the transition portions and the chamber between the inlet end 208 and the outlet end 210 contains no discontinuities that could give rise to pressure waves within the liquid stream flowing through the flow cell body 204.

Turning to FIG. 6, there is shown two sets of images taken of objects in a stream of sea water using the apparatus as shown in FIGS. 1 to 5 and described above. The first set of images on the left of FIG. 6 are of plankton. The second set of images on the right of FIG. 6 are of small bubbles ranging in size from less than 1 to 4.75 mm in the sea water.

Using the apparatus of the present invention, images of objects, such as those of FIG. 6, can be captured from a stream of liquid, such as sea water, flowing through the flow cell at a high flow rate, in particular up to 22 litres per minute or higher.

Finally, turning to FIG. 7, there is shown a representation of an embodiment of an assembly for imaging objects in a liquid stream according to the present invention.

The assembly, generally indicated as 302, comprises an elongate housing 304. The housing 304 is formed from acrylic and is IP67 rated.

A flow cell 310 extends laterally across the housing 304. The flow cell 310 has the general configuration of the flow cell described above and shown in FIGS. 3 and 4. The body of the flow cell 310 extends within the housing 304 and has its inlet end 312 and its outlet end 314 extending through opposing sides of the housing 304. Cam and groove fluid couplings 320 and 322 are provided at the inlet end 312 and outlet end 314 respectively of the housing and connected the flow cell 310 to fluid lines for feeding a liquid stream to and from the flow cell.

The assembly 302 further comprises an illumination apparatus 330 disposed within the housing 304. The illumination apparatus comprises an LED light source 332 and an optical assembly 334 comprising a plate 336 having a pin hole therein and a lens assembly 338. A heat exchanger 340 removes heat produced by the LED light source 332.

The assembly 302 further comprises an imaging apparatus 350 disposed within the housing 304. The imaging apparatus 350 comprises a line scan camera 352. Image data generated by the line scan camera 352 are exported from the assembly by an optical fibre cable 354. The imaging apparatus 350 comprises an optical assembly 356 having a telecentric lens and disposed between the camera 352 and the flow cell 310. A heat exchanger 358 removes heat produced by the camera 352.

The environment within the housing 304, in particular the temperature and humidity are controlled by a Peltier-effect environment control system 360. A fan 362 circulates cool air around the interior of the housing 304.

1-25. (canceled)

26. A flow cell for the analysis of objects in a liquid stream, the flow cell comprising:
a flow cell body having a chamber therein defined by an inner surface of the flow cell body, the flow cell body having an inlet end and an outlet end;
the inlet end of the flow cell body being provided with a first fluid coupling member having a flow passage therethrough;
the outlet end of the flow cell body being provided with a second fluid coupling member having a flow passage therethrough;
the flow cell body comprising a first transparent portion, through which light may enter the chamber to illuminate objects within the chamber, and a second transparent portion through which objects within the chamber may be imaged;
wherein the chamber comprises a first transition portion adjacent the inlet end of the flow cell body, the first transition portion comprising a smooth transition between the flow passage of the first fluid coupling member and the inner surface of the flow cell body defining the chamber; and
wherein the chamber comprises a second transition portion adjacent the outlet end of the flow cell body, the second transition portion comprising a smooth transition between the flow passage of the second fluid coupling member and the inner surface of the flow cell body defining the chamber.

27. The flow cell according to claim 26, wherein the first fluid coupling member and/or the second fluid coupling member comprises a male coupling member for engagement with a corresponding female coupling member.

28. The flow cell according to claim 26, wherein the first fluid coupling member and/or the second fluid coupling member is comprised in a cam and groove fluid coupling.

29. The flow cell according to claim 26, wherein the first and second transparent portions are at the same distance from the inlet end and the outlet end of the flow cell body.

30. The flow cell according to claim 29, wherein the first and second transparent portions are the same distance from both the inlet end and the outlet end of the flow cell body.

31. The flow cell according to claim 26, wherein the first transparent portion is opposite the second transparent portion.

32. The flow cell according to claim 26, wherein a detection zone is defined within the chamber, the detection zone having a rectangular cross-section.

33. The flow cell according to claim 26, wherein the surface of one or both of the first and second transition portions has single facet in both the longitudinal and circumferential directions.

34. The flow cell according to claim 26, wherein the surface of one or both of the first and second transition portions is defined by a smoothed curve function, preferably a polynomial basis function, more preferably a Bezier, Legendre, Chebyshev or Bernstein polynomial function.

35. The flow cell according to claim 26, wherein the first and/or second transition portion extends up to 90% of the distance between the inlet end or outlet end respectively of the flow cell body.

36. An imaging assembly for imaging objects in a liquid stream, the imaging assembly comprising:
a flow cell as defined in claim 26;
an imaging apparatus for imaging objects in the liquid within the detection zone of the flow cell through the second transparent portion of the flow cell body; and
an illumination apparatus for generating light to illuminate a liquid in the detection zone of the flow cell through the first transparent portion of the flow cell body.

37. The imaging assembly according to claim 36, wherein a detection zone is defined within the chamber of the flow cell body, the detection zone of the chamber having a depth that is greater than the depth of focus of the imaging apparatus.

38. The imaging assembly according to claim 37, wherein the depth of the detection zone is from 7 to 15 times the depth of field of the imaging apparatus.

39. The imaging assembly according to claim 36, wherein the imaging apparatus comprises a line scan camera.

40. The imaging assembly according to claim 39, wherein a detection zone is defined within the chamber of the flow cell body and wherein the width of the detection zone perpendicular to the optical axis is no greater than the length of the active line of the line scan camera.

41. The imaging assembly according to claim 36, wherein the imaging apparatus comprises an optical assembly, the optical assembly comprising a telecentric lens.

42. The imaging assembly according to claim 36, wherein the illumination apparatus comprises an optical assembly, the optical assembly comprising a telecentric lens.

43. The imaging assembly according to claim 36, wherein the illumination apparatus comprises a pinhole through which light emitted by the light source is caused to pass.

44. The imaging assembly according to claim 43, wherein the diameter of the pinhole is at least 120% of the optimum diameter of the pinhole.

45. An apparatus for illuminating objects, the apparatus comprising:
a light source for generating light;
a first collimator for collimating the light emitted by the light source, the first collimator comprising a pin hole through which the light from the light source is caused to pass; and
a second collimator comprising a telecentric lens.
